(19) United States
(12) Patent Application Publication
(10) Pub. No.: US 2025/0069754 A1
(43) Pub. Date: Feb. 27, 2025

(54) PREDICTING RISK OF PREGNANCY-RELATED COMPLICATIONS USING MACHINE LEARNING

(71) Applicant: ELYTHEA, INC., San Jose, CA (US)

(72) Inventor: Reetam Ganguli, San Jose, CA (US)

(21) Appl. No.: 18/453,802

(22) Filed: Aug. 22, 2023

(51) Int. Cl. G16H 50/30

(52) U.S. Cl. CPC G16H 50/30

(57) ABSTRACT

A pregnancy risk assessment system predicts risks of various pregnancy-related complications based on prepartum pregnancy factors. One or more machine learning models are trained on a training dataset that includes a range of pregnancy-related statistics. The training dataset is divided into positive training datasets in which a pregnancy-related complication occurred and negative datasets in which the pregnancy-related complication did not occur. A machine learning algorithm trains a model to classify an input dataset based on statistical similarity to the positive and negative datasets. A user interface may obtain input data via a survey presented to a prepartum pregnancy patient and output risk scores based on application of the one or more machine learning models. The risk scores may enable early intervention to prevent or mitigate pregnancy-related complications.

### BACKGROUND

### Technical Field

The described embodiments relate to predicting risk of pregnancy-related complications.

### Description of the Related Art

Pregnancy-related complications represent one of the most common medical issues affecting women. Examples of pregnancy-related complications may include, for example, conditions such as eclampsia, preeclampsia, gestational diabetes, gestational hypertension, preterm labor, postpartum hemorrhage, or various conditions leading to unplanned cesarean section or intensive care unit (ICU) admission. Early detection of risks for such complications may enable prevention and/or improved treatment.

### BRIEF DESCRIPTION OF THE DRAWINGS

FIG. 1 is an example embodiment of a computing environment associated with predicting risk of pregnancy-related complications.

FIG. 2 is an example embodiment of a backend server for training machine learning models and predicting risk of pregnancy-related complications.

FIG. 3 is an example embodiment of a machine learning module for training one or more machine learning models to predict risk of pregnancy-related complications.

FIG. 4 is an example embodiment of a process for training a machine learning model to predict risk of pregnancy-related complications.

FIG. 5 is an example embodiment of a process for applying a machine learning model to predict risk of pregnancy-related complications.

FIG. 6 includes examples of user interface screens associated with generating predicted risks of pregnancy-related complications.

### DETAILED DESCRIPTION

The Figures (FIGS.) and the following description describe certain embodiments by way of illustration only. One skilled in the art will readily recognize from the following description that alternative embodiments of the structures and methods illustrated herein may be employed without departing from the principles described herein. Reference will now be made to several embodiments, examples of which are illustrated in the accompanying figures. Wherever practicable, similar or like reference numbers may be used in the figures and may indicate similar or like functionality.

A pregnancy risk assessment system predicts risks of various pregnancy-related complications based on prepartum pregnancy factors. One or more machine learning models are trained on a training dataset that includes a range of pregnancy-related statistics. The training dataset is divided into positive training datasets in which a pregnancy-related complication occurred and negative datasets in which the pregnancy-related complication did not occur. A machine learning algorithm trains a model to classify an input dataset based on statistical similarity to the positive and negative datasets. A user interface may obtain input data via a survey presented to a prepartum pregnancy patient and output risk scores based on application of the one or more machine learning models. The risk scores may enable early intervention to prevent or mitigate pregnancy-related complications.

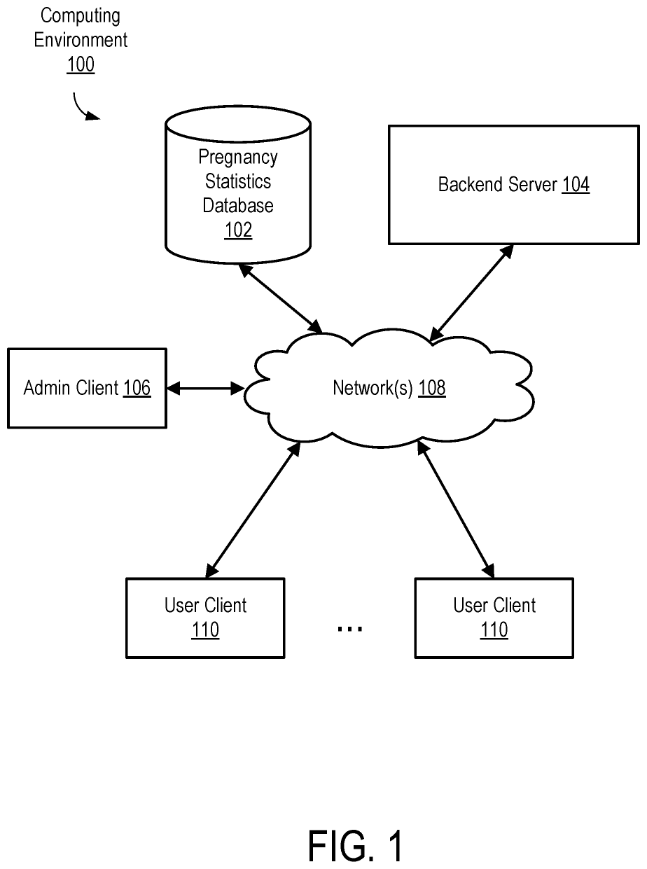

FIG. 1 illustrates an example embodiment of a computing environment 100 for generating risk predictions associated with pregnancy-related complications. The computing environment 100 may include a pregnancy statistics database 102, a backend server 104, an administrative client 106, one or more user clients 110, and a network 108. Alternative embodiments may include different or additional components.

The pregnancy statistics database 102 stores various statistical data associated with pregnancy-related complications. In an embodiment, the pregnancy statistics database 102 may include one or more publicly available datasets such as the Centers for Disease Control and Prevention's (CDC's) National Vital Statistics Live Birth Dataset. The pregnancy statistics database 102 may furthermore include various public or non-public data from electronic health records (EHR) systems, clinical trials, or other sources. The pregnancy statistics database 102 may include one or more cloud-based data sources and/or one or more locally accessible data sources. In some implementations, the pregnancy statistics database 102 may comprise a centralized repository that may aggregate data from multiple different sources. In other implementations, the pregnancy statistics database 102 may refer to two or more disparate data sources that may be managed by different entities and may be independently accessed by the backend server 104. The pregnancy statistics database 102 may be accessible via an application programming interface (API) or may enable to be downloaded via a web browser or other application.

The pregnancy statistics database 102 may incorporate various structured or unstructured information. For example, structured information may include data organized into predefined fields while unstructured data sources may include articles or other media. Examples of structured data fields in the pregnancy statistics database 102 may include, for example, demographic characteristics of the mothers and/or fathers (e.g., race, age, marital status, educational attainment, live birth order, birth interval, etc.), medical and public service utilization (e.g., prenatal care, food during pregnancy, obstetric procedures, labor and delivery characteristics, place of birth, time of birth, attendant at birth, method of delivery, payment source, etc.), maternal behavior and health characteristics (e.g., mother's body mass index, mother's height, mother's pre-pregnancy weight, mother's weight at delivery, weight gain during pregnancy, cigarette smoking before and during pregnancy, risk factors during pregnancy, infections present and/or treated during pregnancy, maternal morbidity, etc.), infant health characteristics (e.g., period of gestation, birthweight, Apgar score, 5-minute score, 10-minute score, abnormal conditions, congenital anomalies, plurality of birth, breastfeeding information, etc.), or other data that may be relevant to analyzing pregnancy-related complications. The pregnancy statistics database 102 may furthermore store various contextual information relating to the quality of data, sources of the data, and computation methods.

The pregnancy statistics database 102 may be organized into records that each correspond to a specific pregnancy instance and include values associated with different fields representing different features. Each data record is not necessarily complete. Some records may include values for every field, while other records may be missing information for one or more fields. Values associated with different fields may be represented in different ways for different fields. For example, data may be presented as numeric values, text values, dates, or coded values. Here, coded values may be stored as a code (e.g., a number) that maps to a definition. For example, the values for a “birthplace” field may comprise codes where “1” represents “Hospital”, “2” represents “Freestanding Birth Center”, etc. Some fields may furthermore utilize a field-specific code to represent an unknown value for that field. The field-specific code for an unknown value may be different for different fields (e.g., the code “9” may represent “Unknown” for the “Birth Place” field, while the code “8” may represent “Unknown” for the “Mother's Race” field.

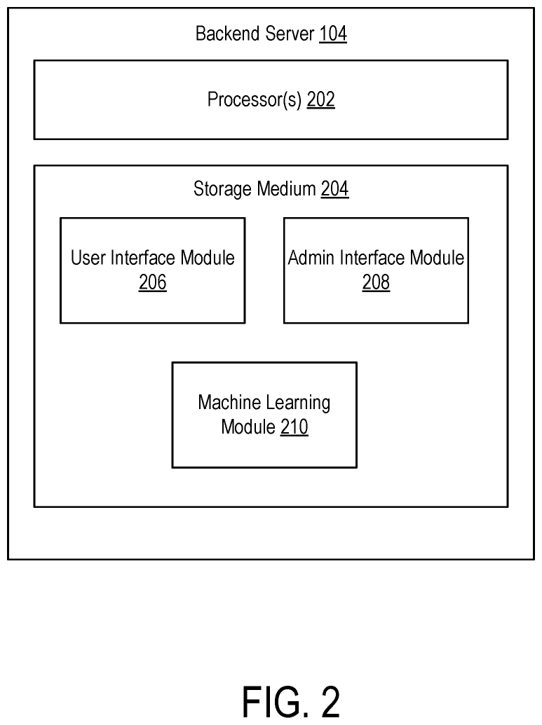

The backend server 104 facilitates training of machine learning models and generating risk predictions associated with pregnancy-related complications based on the machine learning models. In an embodiment, the backend server 104 trains one or more machine learning models based on a training dataset from the pregnancy statistics database 102. The backend server 104 may further apply the one or more machine learning models to an input dataset associated with a pregnant mother to predict risks of one or more pregnancy-related complications occurring in association with the pregnancy. The backend server 104 may also communicate with a front end user interface accessible to the one or more user clients 110 to facilitate various user interface functions, such as enabling users to input information, request predictions, and view and/or interact with various risk data. Further examples of functions performed by the backend server 104 are described in further detail below with respect to FIGS. 2-5.

The backend server 104 may be implemented using cloud processing and storage technologies, on-site processing and storage systems, virtual machines, other technologies, or a combination thereof. For example, in a cloud-based implementation, the backend server 104 may include multiple distributed computing and storage devices managed by a cloud service provider. The various functions attributed to the backend server 104 are not necessarily unitarily operated and managed, and may comprise an aggregation of multiple servers responsible for different functions of the backend server 104 described herein. In this case, the multiple server may be managed and/or operated by different entities. In various implementations, the backend server 104 may comprise one or more processors and one or more non-transitory computer-readable storage mediums that store instructions executable by the one or more processors for carrying out the functions attributed to the backend server 104 herein.

The user client 110 comprises a computing device capable of executing a user interface enabling access to various functions of the backend server 104 described herein. The user interface may be embodied in an application installed on the client device 110 or may comprise a web-based application accessible via web browser. The user interface of the client device 110 may enable various entry of data for communicating to the backend server 104 and enables viewing and/or interaction with various information obtained from the backend server 104 or directly inputted to the user interface. In various embodiments, the user client 110 may be embodied, for example, as a mobile phone, a tablet, a laptop computer, a desktop computer, a gaming console, a head-mounted display device, or other computing device. The user client 110 may be employed by a pregnant mother (or other interested party such as a medical clinician) to generate personalized predictions or another analysis associated with risks of various pregnancy-related complications as will be described further herein. The user client 110 could furthermore include a computing device linked to an EHR system and/or hospital outreach platform that may enable communication with the backend server 104 via an API.

The administrative client 106 comprises a computing device for facilitating administrative functions associated with operation of the backend server 104. For example, the administrative client 106 may comprise a user interface for performing functions such as configuring parameters associated with various machine learning algorithms, initiating deployment of software updates to the user clients 110, etc. The administrative client 106 may furthermore include a computing device linked to an EHR system that may enable various configuration settings and other administrative functions associated with its integration with the backend server 104. The user interface of the administrative client 106 may be embodied as an application installed on the administrative client 106 or may comprise a web-based application accessible via web browser. In various embodiments, the administrative client 110 may be embodied, for example, as a mobile phone, a tablet, a laptop computer, a desktop computer, a gaming console, a head-mounted display device, or other computing device.

The one or more networks 108 provides communication pathways between the pregnancy statistics database 102, the backend server 104, the administrative client 106, and/or the user clients 110. The network(s) 108 may include one or more local area networks (LANs) and/or one or more wide area networks (WANs) including the Internet. Connections via the one or more networks 108 may involve one or more wireless communication technologies such as satellite, WiFi, Bluetooth, or cellular connections, and/or one or more wired communication technologies such as Ethernet, universal serial bus (USB), etc. The one or more networks 108 may furthermore be implemented using various network devices that facilitate such connections such as routers, switches, modems, firewalls, or other network architecture.

FIG. 2 is a block diagram illustrating an example embodiment of a backend server 104. The backend server 104 includes one or more processors 202 and one or more storage mediums 204. The one or more storage mediums 204 includes various functional modules (implemented as instructions executable by the one or more processors 202) including a user interface module 206, an administrative interface module 208, and machine learning module 210. In alternative embodiments, the backend server 104 may include different or additional modules. The one or more processors 202 and one or more storage mediums 204 are not necessarily co-located and may be distributed (e.g., in a cloud architecture).

The user interface module 206 facilitates server-side functions of a user interface accessible on the user clients 110. In an example operation, the user may input various information via a user interface on the user client 110 that is communicated to the user interface module 106, such as inputs describing a pregnancy state including, for example, demographic information about the mother and/or father (e.g., maternal age, residence status, marital status, maternal education level, paternal age, paternal education level, body mass index, pre-pregnancy weight, maternal height, etc.), clinical history (e.g., history of conditions such as diabetes, heart disease, thyroid disease, etc.), prior pregnancy information (e.g., number of prior pregnancies, history of complications, miscarriages, term lengths, prior pregnancy terminations, prior births now dead, previous Cesarean, previous preterm birth, cigarette usage before pregnancy, pre-pregnancy diabetes, pre-pregnancy hypertension, infertility treatment for current pregnancy, live birth order, etc.), gestational period information (e.g., number of prenatal visits, trimester prenatal care began, sexually transmitted disease information, presence or absence of infections, gestational diabetes, gestational hypertension, cigarette usage in each trimester, expected induction of labor, expected augmentation of labor, expected steroid administration, expected antibiotic administration, expected route of delivery, etc.) child development information associated with past pregnancies (e.g., birth defects, child development conditions, etc.), prepartum pregnancy factors affecting the mother (e.g., sickness, mood, pain, etc.), or other data.

In some embodiments, the input data may be obtained interactively by presenting a series of questions via the user interface that enable structured input of data. Questions may be presented for various input forms such as multiple choice, true/false, or text-based inputs. The user interface may utilize various input elements such as radio buttons, drop-down lists, multi-select checkboxes, or freeform text boxes. In an embodiment, the user interface module 106 may facilitate presentation of various feedback during data collecting, such as indicating how far along the user is in the process and/or providing error information for inputs received in an invalid format.

In other embodiments, pregnancy data may be imported from another data source and is not necessarily input via the user interface module 106 of the user client 110. For example, patient data may be obtained from an electronic health records (EHR) system that stores records associated with the individual.

The user interface module 206 may furthermore facilitate presentation of analytical data on the user client 110 in various forms. For example, the user interface module 206 may output a summary of predicted risk scores associated with one or more pregnancy-related complications. The scores may be in the form of a percentage risk likelihood, a numerical score (e.g., on a scale of 1 to 10), a risk category (e.g., high, medium, low), or a combination thereof. In an embodiment, the user interface of the user client 110 may furthermore present various recommendations or contextual information associated with the risk scores. For example, if an individual is predicted to be high risk for eclampsia, the user interface may present links to medical information explaining eclampsia symptoms, diagnosis, treatment, risk factors, and prognosis. Outputs presented by the user interface module 106 may furthermore be color coded based on the risk category or otherwise visually distinguished.

In various embodiments, the user interface module 206 may be designed for direct use by a pregnant mother (or her family), or may be designed for use by a medical practitioner. In some implementations, different instances of the user interface module 206 may be presented depending on the role of the user (e.g., patient, doctor, nurse, pharmacist, etc.)

Although the user interface module 206 is illustrated as component of the backend server 104 in FIG. 2, all or a subset of the functions of the user interface module 206 may instead be executed on the user client 110. For example, the user client 110 may download an application from the backend server 104 that includes all or some of the functions of the user interface module 206. The user client 110 may locally execute instructions associated with these functions.

The administrative interface module 208 facilitates various administrative functions associated with operation of the backend server 104. For example, the administrative interface module 208 may present an interface that enables configuration of various parameters of the machine learning module 210 (described below), controls versions and/or access to applications for the user clients 110, or performs other administrative functions. The administrative interface module 208 may furthermore access, aggregate, and present various statistical data associated with operation of the machine learning module 210 to enable the machine learning models to be evaluated and improved.

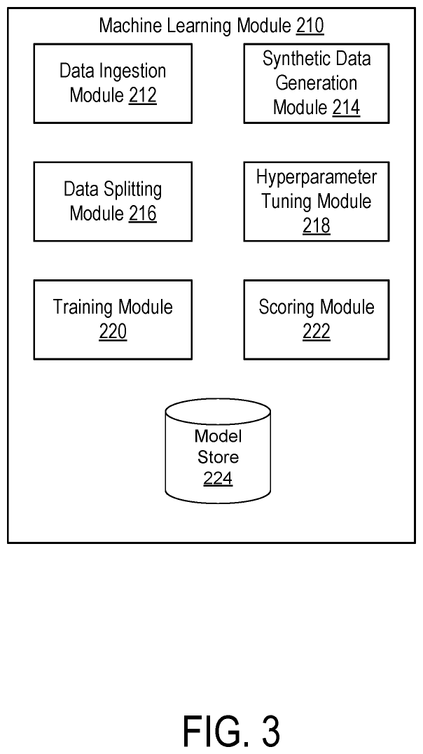

The machine learning module 210 trains one or more machine learning models and applies the trained models to predict risks of various pregnancy-related complications. The predictions may beneficially enable intervention to prevent a high-risk complication from occurring, or may enable improved preparedness for treatment associated with a high-risk complication. An example embodiment of a machine learning module 210 is described in further detail below with respect to FIG. 3.

FIG. 3 is a block diagram illustrating an example embodiment of a machine learning module 210. The machine learning module 210 comprises a data ingestion module 212, a synthetic data generation module 214, a data splitting module 216, a hyperparameter tuning module 218, a training module 220, a scoring module 222, and a model store 224. In alternative embodiments, the machine learning module 210 may comprise different or additional modules. Furthermore, although FIGS. 2-3 describe the machine learning module 210 as a component of the backend server 104, alternative implementations may involve all or a subset of the functions of the machine learning module 210 being executed on the user client 110 or another device instead of the backend server 104.

The data ingestion module 212 obtains training data from the pregnancy statistics database 102 and may perform various pre-processing operations. For example, the data ingestion module 212 may interpret and transform data from the pregnancy statistics database 102 into a standardized format. An example format is as a tabular format (e.g., a comma separated value (.csv) format or other format readable by a spreadsheet application) in which each row corresponds to a record and each column corresponds to a different field (or vice versa). The data ingestion module 212 may furthermore perform various transformations of the values for different fields, such as, for example, converting codes corresponding to unknown values (which may be different codes for different fields) to a common value (such as a zero or null value) for all fields. The data ingestion module 212 may apply various filters to limit the fields to a predefined subset of fields. For example, the data ingestion module 212 may apply a filter to limit point of care fields to those relevant to early-stage pregnancy while excluding point of care fields relating to late stage or post-partum fields that may not be useful for making predictions at early stages of pregnancy. In an embodiment, the data ingestion module 212 may be implemented by applying a set of predefined processing rules to the training dataset. If the training dataset is derived from multiple different data sources with different native formatting, the data ingestion module 212 may apply different rule sets to the different data sources.

The synthetic data generation module 214 may generate synthetic data records to supplement the actual records from the pregnancy statistics database 102. In this technique, the synthetic data generation module 214 generates records that have feature distributions with sufficient similarity to the feature distributions of the actual records. Thus, the synthetic data records may appear to be realistic representations even though they do not directly correspond to actual observations. In an embodiment, the synthetic data may be generated such that it captures intervariable correlations and feature distributions of a minority positive class of data records in which a relatively rare pregnancy-related complication of interest occurred. For example, the synthetic data may be generated such that the positive class of synthetic data records has a similar principal component analysis (PCA) as the positive of class of real data records for the associated pregnancy-related complication of interest. This approach to synthetic data generation may improve learning and adjusting of weights for the minority positive class when training the model. Synthetic data can also be used to generate synthetic records associated with other minority classes like race and/or for the actual outcome. The synthetic data generation module 214 may employ various techniques such as utilizing neural networks or other machine learning models trained to generate synthetic data from a real dataset. The synthetic data may be generated (or post-processed) to be in the same format as the preprocessed data derived from the pregnancy statistics database 102 and may be combined into an aggregate training dataset. In some embodiments, the synthetic data generation module 214 may be employed selectively for training certain machine learning models while not necessarily used in all models. In other embodiments, the synthetic data generation module 214 may be omitted.

The data splitting module 206 splits the training dataset (which may include the pre-processed data from the data ingestion module 212 and/or the synthetic data from the synthetic data generation module 214) into positive and negative training datasets. Here, the positive training dataset comprises the subset of records in which a particular pregnancy-related complication occurred, and the negative training dataset comprises the subset of records in which the particular pregnancy-related complication did not occur.

The data splitting module 206 may separately filter the positive training dataset and the negative training dataset according to different filter parameters. For example, in one implementation, the data splitting module 206 discards records from the negative training dataset that do not include complete information for a predefined set of fields (i.e., records are discarded if any values in the predefined set of fields are unknown). The data splitting module 206 retains all records in the positive training dataset, even if they are incomplete. In an alternative implementation, the data splitting module 206 may retain all records in the negative training dataset regardless of whether they include incomplete information. Furthermore, in one implementation, the data splitting module 206 may filter the positive and negative training datasets according to different data ranges, such that the records in the positive training dataset cover a wider data range than the records in the negative training dataset. Applying more restrictive filters to the negative training dataset than the positive training dataset may beneficially reduce the difference in the respective record counts associated with pregnancy-related complications that occur relatively rarely. In an embodiment, the data splitting module 206 may generate multiple split datasets, where each pair of positive and negative datasets correspond to a different pregnancy-related complication observed in the training dataset.

The training module 220 trains one or more machine learning models based on the positive and negative training datasets. For example, the training module 220 may apply a supervised machine learning algorithm to the positive and negative datasets to learn a set of model parameters (e.g., weights) for classifying a set of input features based on respective statistical similarities to the positive and negative datasets. The classification result may be expressed in terms of a likelihood value (e.g., a value between [0, 1]). Here, a higher likelihood value (representing higher statistical similarity to the positive dataset) represents a higher predicted risk of the pregnancy-related complication occurring. In example implementation, the training module 220 may apply an eXtreme Gradient Boosting (XGB) algorithm to learn the model parameters. In other implementations, algorithms such as logistic regression, random forest, lightGBM, adaboost, neural networks (such as fully connected neural nets, RNNs, CNNs, LSTMs, GRUs, and chained combinations (with multiple layers of different types) may be employed. Furthermore, in some embodiments, the training module 220 may train a stacked machine learning model in which models of different type are trained and stacked together. For example, a stacked classifier may include a combination of an XGB model, logistic regression model, and a random forest meta-classifier.

In an embodiment, the training module 220 may periodically retrain the one more machine learning models as additional training data becomes available. The training module 220 may train different machine learning models for each different potential pregnancy-related complication based on their respective split datasets.

The hyperparameter tuning module 218 may facilitate hyperparameter tuning associated with the machine learning algorithm applied by the training module 220. The hyperparameters may comprise various settings that may affect the outcome of the training process such as, for example, general parameters relating to the type of boosting (e.g., selection of tree or linear model), number of threads, validation settings, etc., booster parameters that may be specific to the selected booster (e.g., step size shrinking, minimum loss reduction, maximum tree depth, sampling method variables, tree method variables, updater variables, etc. weight scaling factor, etc.), learning task parameters that affect learning (e.g., objective function, evaluation metric, etc.), and command line parameters that relate to specific behaviors of the learning algorithm (e.g., number of rounds, path variables, etc.)

In one implementation, the hyperparameter tuning tunes the weight scaling factor in a manner that compensates for imbalances in the counts of positive and negative records in the training dataset. Here, the hyperparameter tuning module 218 may tune the weight scaling factor such that weights applied to the positive training dataset are scaled proportionally to a ratio of the number of records in the negative training dataset to the number of records in the positive training dataset. For example, if the training datasets associated with predicting hemorrhage complications include a 10% occurrence rate and 90% non-occurrence rate, the weights associated with the positive training dataset may be scaled by a factor of 9.

In a further embodiment, the hyperparameter tuning module 218 may perform a hyperparameter optimization by training multiple machine learning models on the same training dataset while varying one or more hyperparameters. Performance of the different models may then be tested on a validation dataset to determine which set of hyperparameters achieves highest performance.

The model store 224 stores the one or more machine learning models generated by the training module 220. As described above, different models may be generated for predicting different pregnancy-related complications and/or for making predictions at different pregnancy stages. Furthermore, some models may be multi-stage models that combine two or more different types of classifiers or other machine learning models.

The scoring module 222 applies the one or more machine learning models to an input feature set to generate scores indicative of the predicted risks of pregnancy-related complications. The risk scores may comprise the likelihood values (e.g., expressed as a percentage or converted to a score on a predefined scoring scale), a classification of the likelihoods between different risk categories (e.g., low risk/high risk), or a combination thereof.

In an embodiment, the scoring module 222 may determine optimal thresholds for classifying likelihoods between high risk and low risk classifications. For example, a Youden's J Statistical Test or other statistical test may be employed to determine a measure of performance associated with different thresholds based on their respective rates of false positive and false negative results in a validation dataset. The thresholds may then be set to optimize performance. Here, the resulting classifications may employ a threshold between a “high risk” and “low risk” classification that is not necessarily 50%. For example, when classifying a risk of eclampsia, a threshold of 35% may be set where a likelihood value over 35% is classified as “high risk” and a likelihood value below 35% is classified as “low risk.”

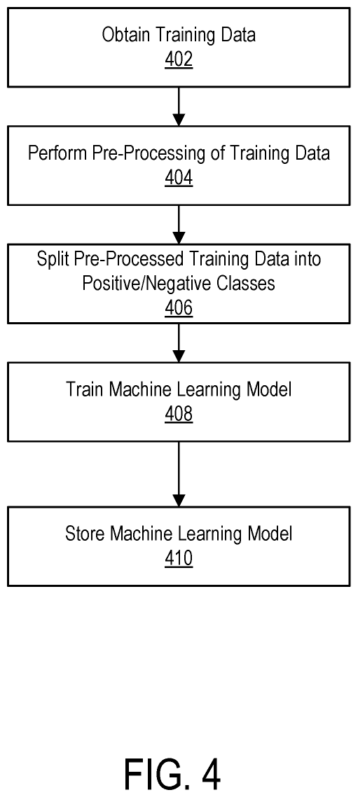

FIG. 4 is a flowchart illustrating an example embodiment of a process for training one or more machine learning models associated with predicting risks of pregnancy-related complications in a prepartum patient. The machine learning module 210 obtains 402 a training data set for a set of pregnancy instances in a training set of patients. The training data set includes information about the training set of patients and the pregnancy instances, and indicates occurrences of a medical event indicative of a pregnancy-related complication. For example, the training data may track occurrences and non-occurrences of medical events such as postpartum hemorrhage, eclampsia, gestational hypertension, pre-eclampsia, NICU admissions, cesarean section, intensive care unit admission, non-adherence to exclusive breastfeeding, preterm labor at a point of care, or other pregnancy-related complications as described elsewhere herein. The training set may furthermore include information such as demographic variables, clinical history information, and gestational period information. In some embodiments, the training data set may be sourced from one or more pregnancy statistics databases 102. Furthermore, the historical data may be supplemented with synthetic data. For example, in one approach, a machine learning model is applied that is trained to generate synthetic data having feature distributions that meet a similarity metric relative to a set of real training data as described above.

The machine learning module 210 performs 404 pre-processing of the training data. In an embodiment, the machine learning module 210 may employ a rule-based approach to pre-processing based on a set of predefined processing rules. For example, the machine learning module 210 may normalize data records from the training set and convert the records to a standardized tabular format. Pre-processing may also include identifying values corresponding to missing or unknown data (e.g., based on field codes that may be different for different fields), and replacing the identified values with a standard value (e.g., zero or a null string) that represents missing or unknown value across the different data fields.

The machine learning module 210 splits 406 the pre-processed training data set into a positive dataset including a subset of records that are positive for the medical event (representing a positive class) and a negative dataset that includes a subset of records that are negative for the medical event (representing a negative class). In one approach, the machine learning module 210 may apply different filters to the positive and negative datasets. For example, the machine learning module 210 filters the negative dataset to remove records that have a missing value for at least one of a predefined set of data fields. The machine learning module 210 may furthermore filter the records to limit the negative dataset to a predefined date range. For the positive class, the machine learning module 210 may include all records even if they have missing values in the predefined set of data fields, and may include records in an extended date range that is wider than the predefined date range used for the negative dataset.

The machine learning module 210 trains 408 a machine learning model based on the positive dataset and the negative dataset to enable classification of an input feature set. The training process may include tunning of various hyperparameters applied in the machine learning algorithm. For example, the machine learning module 210 may tune a weight scaling hyperparameter such that a ratio of scaling weights applied to the positive dataset relative to the negative dataset size is in inverse proportion to the ratio of the size of the positive dataset to the size of negative dataset. In a further embodiment, the machine learning module 210 iteratively applies a machine learning algorithm to the training dataset using varying hyperparameters and evaluates the performance of the resulting models (e.g., in relation to a validation dataset). The hyperparameters may then be optimized based on the performance results and the highest performing model may be selected. In an embodiment, the machine learning model 210 may furthermore adjust one or more thresholds associated with classifying risk dependent on a statistical performance measure.

The machine learning module 210 stores 410 the one or more machine learning models. In an embodiment, the machine learning module 210 may repeat the process of FIG. 4 to train and store multiple machine learning models for predicting different respective medical events. In further embodiments, the machine learning module 210 may train different machine learning models for generating predictions at different pregnancy stages such as during early-stage pregnancy, middle stage pregnancy, and late-stage pregnancy.

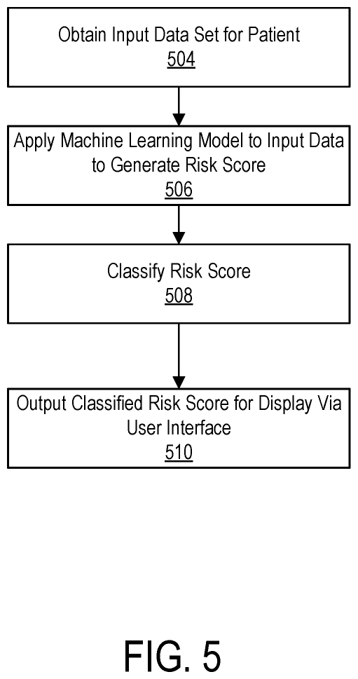

FIG. 5 is a flowchart illustrating an example embodiment of a process for predicting risks of pregnancy-related complications based on one or more trained machine learning models. The machine learning module 210 obtains 504 input data associated with a patient. In one approach, the input data may be obtained via a set of prompts (e.g., a survey) presented to a user via the user client 110. Alternatively, the machine learning module 210 may obtain the input data from a medical practitioner, from an API of an EHR system, or via a combination of data sources. The machine learning module 210 applies 506 one or more machine learning models to the input data to generate one or more risk scores associated with one or more pregnancy-related complications. In an embodiment, multiple machine learning models may be applied to predict respective risk scores associated with different pregnancy-related complications. The machine learning module 210 furthermore classifies 508 the one or more risk scores and outputs 510 the classified risk scores (e.g., for display via a user interface). Here, risk scores expressed as a percentage, as a value on a predefined scale (e.g., 1 to 10), or other likelihood value may be compared to predefined thresholds to classify the risk scores. For example, risk scores may be classified as low risk or high risk. The outputs may be color-coded based on the respective classifications. In another embodiment, the machine learning module 210 may output a recommendation associated with the pregnancy-related complication (e.g., a further diagnostic test, a treatment, a preventative measure, etc.) instead of or in addition to the likelihood value and/or risk classification. The output may optionally be sent to a patient monitoring platform associated with the patient (e.g., a platform accessible by the patient's doctor or other medical practitioner).

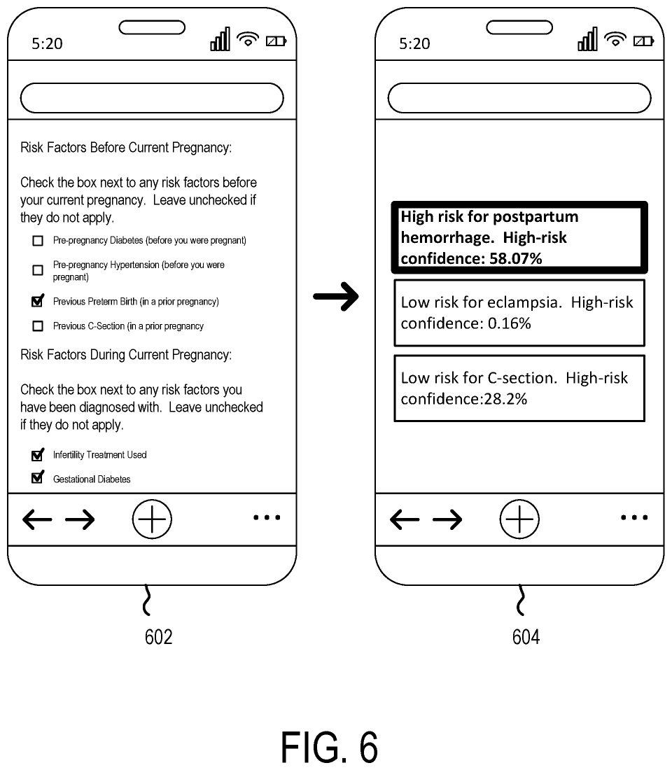

FIG. 6 illustrates example embodiments of user interface screens that may be presented via the user client 110. Interface screen 602 illustrates an example presentation of a portion of a survey for obtaining an input dataset for a patient. In this example, multi-select inputs are presented for various information categories. As described above, the survey may include different types of input formats or a combination of inputs. Interface screen 604 illustrates an example set of results generated for a patient. In this example, the predictions indicate a high risk for postpartum hemorrhage and low risks of eclampsia and C-section. The high-risk classifications may be visually distinguished via color coding, highlighting, animations, or other visual indicators. The illustrated example also shows the specific risk score associated with the different pregnancy-related complications. In alternative variations, the user interface could only show a less granular analytical output such as the risk classification (e.g., high or low), or a tailored consideration (“consider preparing blood and type crossing patient”) without necessarily outputting a specific risk score.

Embodiments of the described computing environment 100 and corresponding processes may be implemented by one or more computing systems. The one or more computing systems include at least one processor and a non-transitory computer-readable storage medium storing instructions executable by the at least one processor for carrying out the processes and functions described herein. The computing system may include distributed network-based computing systems in which functions described herein are not necessarily executed on a single physical device. For example, some implementations may utilize cloud processing and storage technologies, virtual machines, or other technologies.

The foregoing description of the embodiments has been presented for the purpose of illustration; it is not intended to be exhaustive or to limit the embodiments to the precise forms disclosed. Persons skilled in the relevant art can appreciate that many modifications and variations are possible in light of the above disclosure.

Some portions of this description describe the embodiments in terms of algorithms and symbolic representations of operations on information. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a tangible non-transitory computer readable storage medium or any type of media suitable for storing electronic instructions and coupled to a computer system bus. Furthermore, any computing systems referred to in the specification may include a single processor or may be architectures employing multiple processor designs for increased computing capability.

Finally, the language used in the specification has been principally selected for readability and instructional purposes, and it may not have been selected to delineate or circumscribe the inventive subject matter. It is therefore intended that the scope is not limited by this detailed description, but rather by any claims that issue on an application based hereon. Accordingly, the disclosure of the embodiments is intended to be illustrative, but not limiting, of the scope of the invention, which is set forth in the following claims.

1. A method for predicting risk of one or more pregnancy complications of a prepartum pregnancy patient, the method comprising:
obtaining a training data set for a set of pregnancy instances in a training set of patients, the training data set including information about the training set of patients and the pregnancy instances, and the training data set indicating occurrences of a medical event associated with the pregnancy instances;
pre-processing the training data set according to a set of pre-processing rules to generate a pre-processed training data set;
splitting the pre-processed training data set into a positive dataset that represent a positive class positive for the medical event and a negative dataset that represent a negative class that are negative for the medical event;
applying a machine learning algorithm to the positive dataset and the negative dataset to train a machine learning model to perform classification of an input feature set between the positive class and the negative class;
storing the machine learning model;
obtaining prepartum pregnancy information associated with a test patient via a user interface of a client device;
applying the machine learning model to predict a likelihood of the medical event based on the prepartum pregnancy information;
generating an analytical output based on the likelihood; and
outputting the analytical output to the user interface of the client device.

2. The method of claim 1, wherein pre-processing the training data set comprises:
normalizing entries in the training data set into a standardized tabular format.

3. The method of claim 1, wherein pre-processing the training data set comprises:
identifying, for different data fields in the training data set, respective values in the different data fields that represent missing values; and
replacing each of the respective values to a standard value across the different data fields.

4. The method of claim 1, wherein splitting the training dataset comprises applying different filtering rules to the positive dataset and the negative dataset, wherein filtering rules applied to the negative dataset are more restrictive in at least one aspect than filtering rules applied to the positive dataset.

5. The method of claim 1, wherein splitting the training data set comprises:
applying a first filter to identify first samples corresponding to the negative class in which the medical event did not occur, wherein the first filter removes samples that have a missing value for at least one of a predefined set of data fields, and wherein the first filter removes samples associated with a date outside a predefined time range;
applying a second filter to identify samples corresponding to the positive class in which the medical event occurred, wherein the second filter allows samples having missing values in the predefined set of data fields, and wherein the second filter allows samples outside the predefined time range.

6. The method of claim 1, wherein applying the machine learning algorithm comprises:
tuning a weight scaling of the machine learning algorithm to scale a weight for applying to the positive class relative to a weight for applying to the negative class inversely to a size of the positive class relative to a size of the negative class.

7. The method of claim 1, wherein applying the machine learning algorithm comprises:
iteratively applying the machine learning algorithm and evaluating performance of the machine learning model while varying hyperparameters of the machine learning algorithm to determine optimized hyperparameters.

8. The method of claim 1, wherein obtaining the training data set comprises:
obtaining real training data associated with real patients;
applying a machine learning model to generate synthetic data having feature distributions meeting a similarity metric relative to the real training data; and
including the real training data and the synthetic data in the training data set.

9. The method of claim 1, wherein applying the machine learning algorithm comprises:
generating a statistical measure of performance of the machine learning model; and
adjusting, based on the statistical measure, a threshold for classifying a risk assessment based on the likelihood.

10. The method of claim 1, wherein the medical event comprises at least one of: postpartum hemorrhage, eclampsia, gestational hypertension, pre-eclampsia, NICU admissions, cesarean section, intensive care unit admission, non-adherence to exclusive breastfeeding, and preterm labor at a point of care.

11. The method of claim 1, wherein the information about the training set of patients and the pregnancy instances comprises at least one of: demographic variables, clinical history information, and gestational period information.

12. The method of claim 1, wherein splitting the pre-processed training dataset, applying the machine learning algorithm, and storing the machine learning model comprises splitting pre-processing training dataset according to multiple different types of pregnancy-related complications, training different machine learning models for the different types of pregnancy-related complications, and storing the different machine learning models.

13. The method of claim 1, wherein splitting the pre-processed training dataset, applying the machine learning algorithm, and storing the machine learning model comprises splitting pre-processing training dataset according to different data fields that become known at different pregnancy stages, training different machine learning models for making predictions at the different pregnancy stages, and storing the different machine learning models.

14. The method of claim 1, wherein outputting the analytical output comprises:
transmitting the output to a remote patient monitoring platform for monitoring the prepartum pregnancy patient by a medical practitioner.

15. The method of claim 1, wherein outputting the analytical output comprises outputting at least one of a risk classification derived from the likelihood, a recommendation associated with the likelihood, or a link to an information source associated with the pregnancy-related complication.

16. A non-transitory computer-readable storage medium storing instructions for predicting risk of one or more pregnancy complications of a prepartum pregnancy patient, the instructions when executed by one or more processors causing the one or more processors to perform steps including:
obtaining a training data set for a set of pregnancy instances in a training set of patients, the training data set including information about the training set of patients and the pregnancy instances, and the training data set indicating occurrences of a medical event associated with the pregnancy instances;
pre-processing the training data set according to a set of pre-processing rules to generate a pre-processed training data set;
splitting the pre-processed training data set into a positive dataset that represent a positive class positive for the medical event and a negative dataset that represent a negative class that are negative for the medical event;
applying a machine learning algorithm to the positive dataset and the negative dataset to train a machine learning model to perform classification of an input feature set between the positive class and the negative class;
storing the machine learning model;
obtaining prepartum pregnancy information associated with a test patient via a user interface of a client device;
applying the machine learning model to predict a likelihood of the medical event based on the prepartum pregnancy information;
generating an analytical output based on the likelihood; and
outputting the analytical output to the user interface of the client device.

17. The non-transitory computer-readable storage medium of claim 16, wherein pre-processing the training data set comprises:
normalizing entries in the training data set into a standardized tabular format.

18. The non-transitory computer-readable storage medium of claim 16, wherein pre-processing the training data set comprises:
identifying, for different data fields in the training data set, respective values in the different data fields that represent missing values; and
replacing each of the respective values to a standard value across the different data fields.

19. The non-transitory computer-readable storage medium of claim 16, wherein splitting the training dataset comprises applying different filtering rules to the positive dataset and the negative dataset, wherein filtering rules applied to the negative dataset are more restrictive in at least one aspect than filtering rules applied to the positive dataset.

20. A computer system comprising:
one or more processors; and
a non-transitory computer-readable storage medium storing instructions for predicting risk of one or more pregnancy complications of a prepartum pregnancy patient, the instructions when executed by the one or more processors causing the one or more processors to perform steps including:
obtaining a training data set for a set of pregnancy instances in a training set of patients, the training data set including information about the training set of patients and the pregnancy instances, and the training data set indicating occurrences of a medical event associated with the pregnancy instances;
pre-processing the training data set according to a set of pre-processing rules to generate a pre-processed training data set;
splitting the pre-processed training data set into a positive dataset that represent a positive class positive for the medical event and a negative dataset that represent a negative class that are negative for the medical event;
applying a machine learning algorithm to the positive dataset and the negative dataset to train a machine learning model to perform classification of an input feature set between the positive class and the negative class;
storing the machine learning model;
obtaining prepartum pregnancy information associated with a test patient via a user interface of a client device;
applying the machine learning model to predict a likelihood of the medical event based on the prepartum pregnancy information;
generating an analytical output based on the likelihood; and
outputting the analytical output to the user interface of the client device.
